(19) United States
(12) Patent Application Publication
(10) Pub. No.: US 2004/0044260 A1
(43) Pub. Date: Mar. 4, 2004

(54) Method and apparatus for producing styrene by catalytic dehydration of 1-phenylethanol

(76) Inventors: Leslie Andrew Chewter, Amsterdam (NL); Timothy Michael Nisbet, Amsterdam (NL); Joseph Broun Powell, Houston, TX (US)

Correspondence Address: Jennifer D. Adamson, Shell Oil Company, Legal-Intellectual Property, P.O. Box 2463, Houston, TX, 77252-2463, US

(21) Appl. No.: 10/650,640

(22) Filed: Aug. 28, 2003

(30) Foreign Application Priority Data
Aug. 28, 2002 (EP) 02255962.9

(51) Int. Cl. C07C 1/00

(52) U.S. Cl. 585/319; 585/441; 585/469

(57) ABSTRACT

The invention involves a method for producing styrene, using at least two separate reactors, by feeding a 1-phenylethanol-rich reaction mixture to a first reactor, transferring the partially catalytically dehydrated mixture to a second reactor or to a distillation unit, and separating the mixture into a fraction of low-molecular compounds and transporting it to an outlet, and to a fraction containing high-molecular compounds and transporting it to the second reactor, or feeding a part of the catalytically dehydrated mixture to the distillation unit and the other part to the second reactor, optionally recycling a part to the first reactor, and/or optionally transporting a part to another reactor, and/or to the distillation unit or to another distillation unit; provided that part of the reaction mixture of at least one of the reactors is transported to the distillation unit. The invention further pertains to an apparatus for performing this method.

### FIELD OF THE INVENTION

The invention pertains to a method and an apparatus for producing styrene by catalytic dehydration of 1-phenylethanol.

### BACKGROUND OF THE INVENTION

Methods for producing styrene by catalytic dehydration of 1-phenylethanol are known in the art, and various publications describing processes and apparatuses for such process are known. The synthesis of styrene is important because this product functions as a starting material for valuable commercial products such as plastics and the like. In U.S. Pat. No. 3,526,674, 1-phenylethanol is dehydrated to styrene by a catalytic process at a temperature of at least 220° C. According to this process, the feed is fed to a reactor and the vapor product is introduced into a distillation zone. Within this distillation zone, unreacted 1-phenylethanol is separated from the styrene and withdrawn from the bottom of the distillation zone and recycled to the reactor. Although this method provides enhanced selectivity, there is room for improvement in terms of yield of styrene and suppressing the production of heavy side-products.

### SUMMARY OF THE INVENTION

It has now been found that when applying a process with at least two separate reactors, fewer heavy side-products and a higher yield of styrene may be obtained.

The invention relates to a process with at least two separate reactors, comprising a first step of feeding a 1-phenylethanol-rich reaction mixture to a first reactor operating at a temperature between 150° C. and 360° C., and thereafter transferring the partially catalytically dehydrated mixture to a second reactor operating at 150° C. to 360° C., or a distillation unit, to separate the mixture into a fraction comprising low-molecular weight compounds which is transported to an outlet, and to a fraction comprising high-molecular weight compounds which is transported to the second reactor, or feeding a part of the catalytically dehydrated mixture to the distillation unit and the other part to the second reactor, from which a part is optionally recycled to the first reactor, and/or optionally a part is transported to a further reactor, and/or to the distillation unit and/or to another distillation unit; provided that part of the reaction mixture of at least one of the reactors is transported to the distillation unit. The invention also relates to an apparatus for performing this method.

### BRIEF DESCRIPTION OF THE DRAWINGS

FIG. 1 shows a block diagram of an embodiment of the invention wherein the first reactor is in direct contact with the second reactor.

FIG. 2 shows an alternative of FIG. 1 wherein the second reactor is exclusively fed through the distillation column.

FIG. 3 shows an embodiment wherein only the second reactor is in contact with the distillation column.

### DETAILED DESCRIPTION OF THE INVENTION

The invention preferably pertains to the process for preparing styrene by catalytically dehydrating 1-phenylethanol in the liquid phase. Dehydration conditions of temperature and pressure as well as the selection of the catalyst are generally known in the field. Such procedure generally involves dehydrating 1-phenylethanol in the liquid phase at temperatures ranging from about 150° C. to about 350° C., preferably from about 180° C. to about 280° C., and more preferably from about 200° C. to 260° C. The pressure generally is sub-atmospheric to atmospheric, from about 0.05 bar to about 1 bar, preferably from about 0.2 bar to about 0.6 bar, and more preferably from about 0.3 bar to about 0.5 bar.

Acidic type catalysts are preferably employed such as aliphatic and aromatic sulfonic acids. Examples are oxalic acid, sulfuric acid, and particularly p-toluene sulfonic acid.

In a further object, the invention pertains to an apparatus for producing styrene by catalytic dehydration of 1-phenylethanol comprising a feed line to a first reactor with optionally a recycle inlet and further comprising at least one of a conduit to a second reactor and a conduit to a distillation unit, comprising at its upper end an outlet for releasing low-molecular weight compounds and at its lower end a conduit for feeding high-molecular weight compounds into the second reactor (or optionally into the first reactor), comprising at least one inlet for high-molecular compounds, optionally a conduit to the distillation unit or to another distillation unit, and optionally an outlet to a conduit that is connected to the optional recycle inlet of the first reactor and/or a conduit to a further reactor, at least one of the first and second reactor comprising a conduit to the distillation unit.

The apparatus of the invention, with at least two vessels in series, at least one of which is heated, wherein the styrene monomer (and co-product water) formed is vaporized, allows in-situ styrene monomer removal. Because the boiling points of styrene monomer and 1-phenylethanol are relatively close, some further separation of this vapor stream is required. This is achieved by the use of conventional distillation. Ideally, all 1-phenylethanol would be returned from the bottom of the column directly to the reactor, giving 100% of 1-phenylethanol conversion per pass over the combination of reactor and distillation column. However, in a commonly used process for preparing 1-phenyl ethanol, i.e. ethyl benzene to hydroperoxide and then conversion of propene to propene oxide, the component methyl phenyl ketone (MPK; acetophenone), which has an almost identical boiling point as 1-phenylethanol is formed as a side-product. MPK is usually converted to 1-phenylethanol in a hydrogenation unit downstream of the dehydration step, therefore, MPK must pass essentially unconverted through the dehydration reactor. This means that MPK, and hence any unconverted 1-phenylethanol, must be allowed to exit the distillation with the crude styrene.

In one embodiment of this invention, two reactors are operated in series and vapor product from each is passed to a distillation column. This may be classified as a staged reactor train with vapor cross flow. However, the two vapor streams are of different composition: the first reactor vapor stream being richer in 1-phenylethanol. Although the feed may enter the distillation column at the same point, it is preferred to make use of the different compositions by feeding these two streams to the distillation column at different points. The vapor stream from the first reactor preferably enters at a lower stage than the vapor stream from the second reactor, which, being relatively poor in 1-phenylethanol and rich in MPK, should be fed nearer the top of the column. This split feeding should, in principle, allow more 1-phenylethanol to be returned directly from the distillation to the reactor, while achieving the desired bleed of MPK in the crude styrene.

It is also possible to house the separate reactors in one vessel, for instance by using separation walls.

The liquid phase is maintained in the reactor by the presence of heavier components such as styrene oligomers and diphenyl ethyl ethers, which can optionally be recycled from the outlet of the second reactor to the inlet of the first reactor. This ensures a regular flow of the catalyst containing liquid through the two reactors with the option of feeding the second reactor hydraulically from the first.

A bleed from the heavies recycle stream may be fed to other separation means, for instance another stripping column, if so desired, to separate the heaviest components (oligomers), which are taken off via the bottom of this column while 1-phenylethanol and ethers from the top of the column are recycled to the one of the reactors.

The heat requirements, due to the endothermic nature of the reaction and to the heat of evaporation of products, can be satisfied by using conventional heating equipment as is known by the artisan, such as external heat exchangers. The use of internal heat exchange elements is less preferred because of the possibility of fouling. For the same reason, the reaction mixture is preferably circulated through the heat exchanger tube bundle rather than on the outside of the tube bundle.

Because of the back mixing in the individual stages, the reactor set up enables the reaction temperature to be controlled at every stage and thus makes it possible to maintain a constant temperature or a different temperature in each reactor.

An alternative of operating the staged reactor train with vapor cross flow is to operate the reactor train with vapor flow co-current to the liquid. In this case, a single vapor stream is sent to the downstream distillation column. Again, the liquid phase can be maintained by recycle of heavy components from the liquid outlet to the inlet of the reactor, and heat is preferably provided by external heat exchangers.

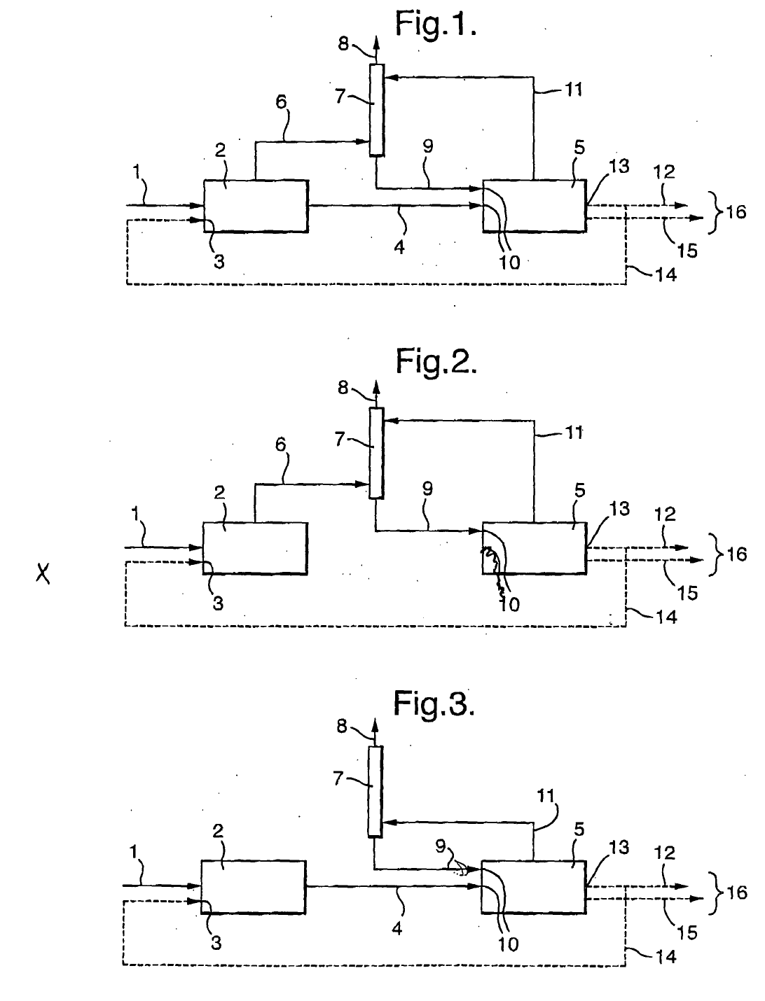

The apparatus according to FIG. 1 comprises a feed line 1 to a first reactor 2 with optionally a recycle inlet 3 and further comprising a conduit 4 to a second reactor 5 and a conduit 6 to a distillation unit 7, comprising at its upper end an outlet 8 for releasing low-molecular weight compounds and at its lower end a conduit 9 for feeding high-molecular weight compounds into the second reactor 5. The second reactor comprises, in this Figure, two inlets 10 for introducing the high-molecular weight compounds, and a conduit 11 to the distillation unit 7. Conduit 11 is optional and may be deleted, if one so wishes. The second reactor may further optionally have a conduit to another distillation unit 12, for instance when conduit 11 is not present, and optionally an outlet 13 to a conduit 14 that is connected to the optional recycle inlet 3 of the first reactor 2. The second reactor may also have an optional conduit 15 to a further reactor 16. In this embodiment, both the first and second reactor comprise a conduit 6, 11, to the distillation unit 7. The reactors may be of the common type as known to the skilled person, for instance a sparged tank, trickle bed, and the like.

In FIG. 2, an alternative of the above embodiment is given. In this embodiment, the first reactor 2 does not have a direct conduit 4 to the second reactor 5, rather the heavy compounds are now transferred to the second reactor via distillation column 7, through conduits 6 and 9. Also in this embodiment, conduit 11 is optional and may be deleted.

In FIG. 3, an embodiment is given wherein conduit 6 from the first reactor 2 to the distillation unit 7 has been deleted. Conduits 4 and 11 are no longer optional in this embodiment.

The advantages of the invention are further illustrated by the following non-limiting examples.

### EXAMPLE 1

In a reactor, air was blown through ethylbenzene. The product comprised ethylbenzene hydroperoxide. This product was mixed with a solution containing sodium hydroxide. The neutralized mixture was subsequently water washed. The product obtained was reacted with propene in the presence of a titania on silica catalyst as described in the Example of EP-A-345856, herein incorporated by reference. Unreacted ethylbenzene and propylene oxide were removed by distillation. The crude 1-phenylethanol remaining after ethylbenzene removal was used as feed for the dehydration reactors.

p-Toluene sulfonic acid was added to the crude 1-phenolethanol stream at a level of 114 ppmw. The stream was then fed continuously to 2 reactors in series, at a rate of 1.9 kg feed per kg liquid hold-up in the reactors per hour. Reactor temperature was 232° C. and the reactor pressure was 0.43 bar. Downstream of the second reactor, vapor and heavy liquid products were separated in a vessel. The heavy products were recirculated to the first reactor and a small bleed applied to keep the amount of heavy products in the system constant. The vapor was sent to the bottom of a distillation column of 5 trays to which reflux was applied. Overhead product was condensed and separated into an organic and aqueous layer. The organic layer was analyzed by gas chromatography to determine the styrene and residual 1-phenylethanol content. Liquid leaving the bottom of the distillation column was recycled to the inlet of the second reactor. The amount of heavy by-products formed was 3.2 wt. % on styrene produced, and 1-phenylethanol conversion was 95.7%.

### Comparative Example 1

A crude 1-phenylethanol stream was prepared in the same manner as in Example 1. p-Toluene sulfonic acid was added to the crude 1-phenolethanol stream at a level of 200 ppmw. The stream was then fed continuously to a single reactor, at a rate of 0.65 kg feed per kg liquid hold-up in the reactor per hour. The reactor contained heavy liquid products of the dehydration reaction. Reactor temperature was 225° C. and the reactor pressure was 0.40 bar. Further heavy liquid products formed were allowed to accumulate in the reactor. The vapor from the reactor was sent to the bottom of a distillation column of 5 trays to which reflux was applied. Overhead product was condensed and separated into an organic and aqueous layer. The organic layer was analyzed by gas chromatography to determine the styrene and residual 1-phenylethanol content. Liquid leaving the bottom of the distillation column was recycled to the reactor. The amount of heavy by-products formed was 7.5 wt. % on styrene produced, and 1-phenylethanol conversion was 95.7%.

### EXAMPLE 2

Based on the kinetics of the reaction of 1-phenylethanol to styrene using p-toluene sulfonic acid as catalyst, and of the subsequent reaction of styrene to polymeric material, the amount of polymeric material formed was calculated for different reactor configurations. The calculations were performed using the flow sheeting software ASPEN Plus 10.2. In the calculations, the reactor feed consists of 85 wt. % of 1-phenylethanol, 12 wt. % of acetophenone (MPK), 2 wt. % of 2-phenylethanol and 1 wt. % of 2,3-diphenylethylether. In all cases, the temperature at the outlet of the last reactor is 240° C., the reactor pressure is 0.2 bar, the catalyst concentration at the outlet of the last reactor is 0.5 wt. % and the feed rate to the system in kg feed per kg liquid hold-up in the reactors per hour is adjusted so that the overall conversion of 1-phenylethanol is 90%. In all cases, heavy liquid products are recycled to the reactor feed at a ratio of 1.67 kg per kg fresh feed. The amount of polymeric material formed from styrene for different reactor configurations is given in Table 1, below:

TABLE 1

| | Amount of polymeric |
|---|---|
| | material formed from |
| | styrene |
| Configuration | (kg/(ton styrene) |
| Single continuous stirred | 10.3 |
| tank (CSTR) with | |
| distillation column | |
| downstream | |
| Two CSTR's in series, | 4.1 |
| with distillation column | |
| downstream of 2nd reactor | |
| Two CSTR's in series. | 2.1 |
| Product of first reactor | |
| is sent to a distillation | |
| column where crude | |
| styrene product is | |
| removed overhead. Bottom | |
| product of distillation | |
| column is sent to the | |
| second CSTR. Product of | |
| the 2nd CSTR is sent to a | |
| second distillation | |
| column | |

What is claimed:

1. A process for producing styrene by catalytic dehydration using at least two separate reactors, said process comprising:
(a) feeding a 1-phenylethanol-rich reaction mixture to a first reactor operating at a temperature between 150° C. and 350° C.; and
(b) transferring the partially catalytically dehydrated mixture to a second reactor operating at a temperature between 150° C. and 350° C.

2. The process of claim 1, in which step (a) is followed by the following steps:
(b) transferring the partially catalytically dehydrated mixture to a distillation unit;
(c) separating the mixture from the distillation unit into a fraction comprising low-molecular weight compounds and high molecular weight compounds;
(d) transporting the fraction comprising low-molecular weight compounds to an outlet; and
(e) transporting the fraction comprising high molecular weight compounds to a second reactor.

3. The process of claim 2, in which step (b) comprises:
(b) transferring part of the catalytically dehydrated mixture to a distillation unit, and part of the mixture to a second reactor operating at a temperature between 150° C. and 350° C.

4. The process of claim 3 wherein step (b) further comprises recycling part of the mixture from the second reactor into the first reactor.

5. The process of claim 3 wherein step (b) further comprises transferring part of the mixture from the second reactor to a third reactor.

6. The process of claim 3 wherein step (b) further comprises transferring part of the mixture from the second reactor to the distillation unit.

7. The process of claim 3 wherein step (b) further comprises transferring part of the mixture from the second reactor to a second distillation unit.

8. The process of claim 1 wherein the dehydration reaction is performed in the liquid phase at a reactor temperature of 180° C. to 280° C.

9. The process of claim 8 wherein a reactor temperature of 200° C. to 260° C. is used.

10. An apparatus for producing styrene by catalytic dehydration of 1-phenylethanol comprising a feed line to a first reactor with optionally a recycle inlet and further comprising at least one of a conduit to a second reactor and a conduit to a distillation unit, comprising at its upper end an outlet for releasing low-molecular compounds and at its lower end a conduit for feeding high-molecular compounds into the second reactor, comprising at least one inlet for high-molecular compounds, optionally a conduit to the distillation unit or to another distillation unit, and optionally an outlet to a conduit that is connected to the optional recycle inlet of the first reactor and/or a conduit to a further reactor, at least one of the first and second reactor comprising a conduit to the distillation unit.

11. The apparatus of claim 10 comprising a first reactor with an optional recycle inlet and a conduit to a second reactor, comprising a conduit to the distillation unit and optionally an outlet to the conduit that is connected to the optional recycle inlet of the first reactor.
